(19) United States
(12) Patent Application Publication
(10) Pub. No.: US 2015/0010441 A1
(43) Pub. Date: Jan. 8, 2015

(54) Electronic Pregnancy Test Device

(71) Applicant: Elana M. Kahn, Brighton, MA (US)

(72) Inventor: Elana M. Kahn, Brighton, MA (US)

(21) Appl. No.: 13/933,393

(22) Filed: Jul. 2, 2013

(51) Int. Cl. G01N 33/76

(52) U.S. Cl. CPC G01N 33/76
USPC 422/403

(57) ABSTRACT

A handheld, readily portable, electronic pregnancy test device including a Central Processing Unit in operational communication with a human Chorionic Gonadotropin hormone sensor able to detect a level of human Chorionic Gonadotropin in a blood sample disposed upon a test strip, said test strip loaded into the device through a test strip portal, wherein a pregnancy is verifiable and human Chorionic Gonadotropin doubling times are immediately calcuble whereby the health of the pregnancy is determinable absent direct medical assistance.

### CROSS-REFERENCE TO RELATED APPLICATIONS

Not Applicable

### FEDERALLY SPONSORED RESEARCH OR DEVELOPMENT

Not Applicable

### INCORPORATION BY REFERENCE OF MATERIAL SUBMITTED ON A COMPACT DISK

Not Applicable

### BACKGROUND OF THE INVENTION

Various types of pregnancy test devices are known in the prior art. However, what is needed is a handheld, readily portable, electronic pregnancy test device that includes a Central Processing Unit in operational communication with a human Chorionic Gonadotropin hormone sensor able to detect a level of human Chorionic Gonadotropin in a blood sample disposed upon a test strip and loaded into the device through a test strip portal, wherein a pregnancy is verifiable and human Chorionic Gonadotropin doubling times are calcuble whereby the health of the pregnancy is determinable absent direct medical assistance.

### FIELD OF THE INVENTION

The present invention relates to an electronic pregnancy test device, and more particularly, to a handheld, readily portable, electronic pregnancy test device that includes a Central Processing Unit in operational communication with a human Chorionic Gonadotropin hormone sensor able to detect a level of human Chorionic Gonadotropin in a blood sample disposed upon a test strip and loaded into the device through a test strip portal, wherein a pregnancy is verifiable and human Chorionic Gonadotropin doubling times are calcuble whereby the health of the pregnancy is determinable absent direct medical assistance.

### SUMMARY OF THE INVENTION

The general purpose of the electronic pregnancy test device, described subsequently in greater detail, is to provide an electronic pregnancy test device which has many novel features that result in an electronic pregnancy test device which is not anticipated, rendered obvious, suggested, or even implied by prior art, either alone or in combination thereof.

The present electronic pregnancy test device not only tests for the presence of human Chorionic Gonadotopin (“hCG”), a hormone signifying pregnancy; the present device enables handheld monitoring of hCG levels in a patient and calculates doubling times of hCG whereby abnormalities in the pregnancy may be detected (such as, for example, ectopic pregnancy or miscarriage) without the patient having to repeatedly travel to a medical practitioner for blood work.

The present electronic pregnancy test device reads a sample of blood positioned upon a test strip, which test strip is subsequently loaded into a test strip portal disposed on the side of the device, wherein an hCG sensor detects the level of hCG in the blood sample and stores said data in a read-write memory controlled and accessed by means of a Central Processing Unit (“CPU”).

The present device is contemplated to calculate hCG doubling times whereby a pregnancy may be monitored to provide information as to the health of the pregnancy. Data may be input into the device whereby the current term of pregnancy may be recorded. Moreover, from at least two readings of relevant blood samples, the device may display an estimate of the phase and/or trimester the pregnancy has reached, and an estimated birthday therefrom. The device may also include warnings of irregularities in detected relative levels of hCG, whereby medical attention should be sought.

The present device, therefore, includes a Light Emitting Diode (“LED”) display upon which data is rendered and blood results displayed. A blood sample from a finger tip is drawn onto a test strip and then positioned upon a test strip support disposed extending from the device. The test strip support is moveable from an extended position, extending from the device, to an internal position, loaded into the device through a test strip portal. A raised member is disposed upon the test strip support which abuts endwise the test strip there positioned. The raised member also activates the hCG sensor when the test strip support is moved to the internal position and loaded into the device.

A plurality of control buttons disposed upon the device enables interaction and control of data. The plurality of control buttons also enables selection of data, as desired. The present electronic pregnancy test device includes a Central Processing Unit (“CPU”) disposed in operational communication with the plurality of control buttons, the hCG sensor, and the LED display, and hCG doubling times are calculable and the pregnancy phase, and any detected potential abnormality of said pregnancy, are communicable by means of the device.

The present electronic pregnancy test device is contemplated to be handheld and readily portable. Use of the device obviates frequent trips to the doctor for blood work while trying to verify pregnancy, and enables regular checks and monitoring in the home, whereby direct medical supervision may be lessened over the course of a pregnancy in the absence of irregular or abnormal results.

Thus has been broadly outlined the more important features of the present electronic pregnancy test device so that the detailed description thereof that follows may be better understood and in order that the present contribution to the art may be better appreciated.

Objects of the present electronic pregnancy test device, along with various novel features that characterize the invention are particularly pointed out in the claims forming a part of this disclosure. For better understanding of the electronic pregnancy test device, its operating advantages and specific objects attained by its uses, refer to the accompanying drawings and description.

### BRIEF DESCRIPTION OF THE DRAWINGS

### Figures

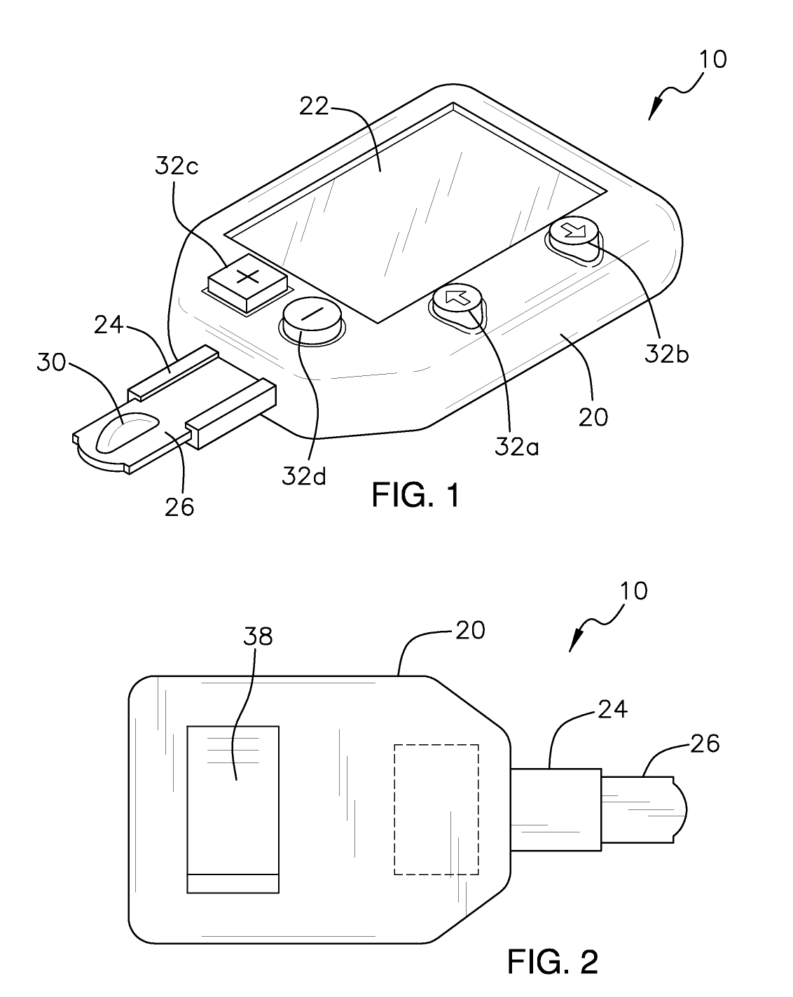

FIG. 1 is an isometric view.

FIG. 2 is a bottom view.

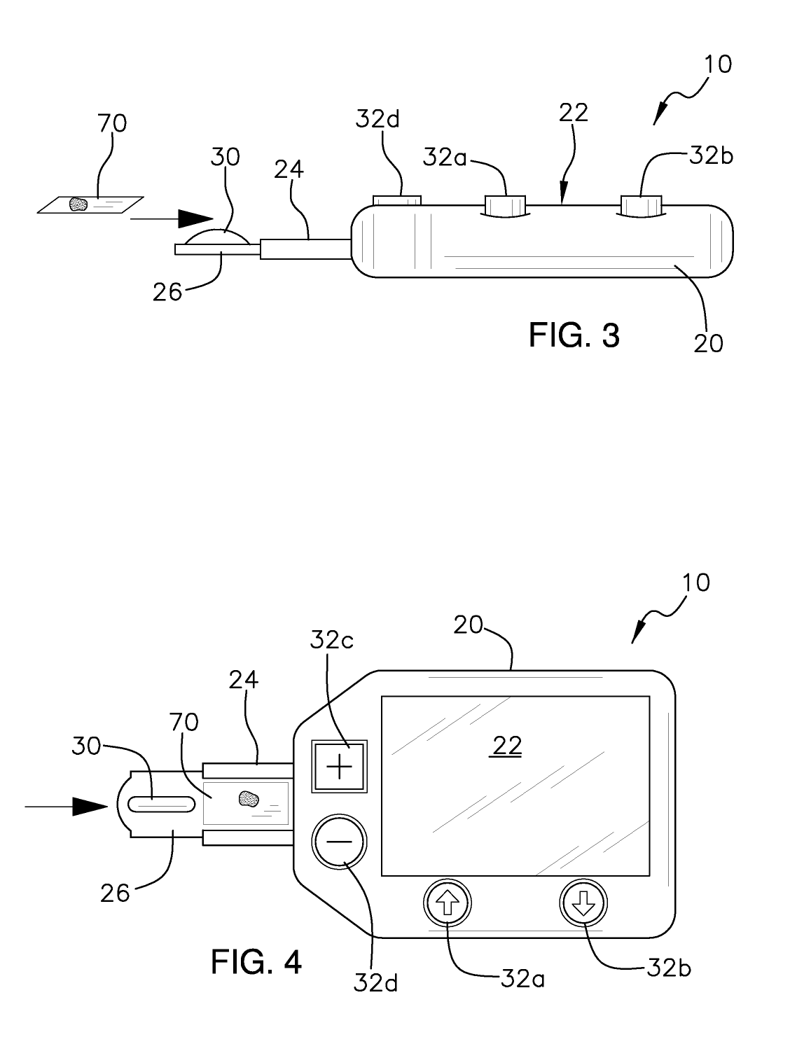

FIG. 3 is a side view.

FIG. 4 is a top view.

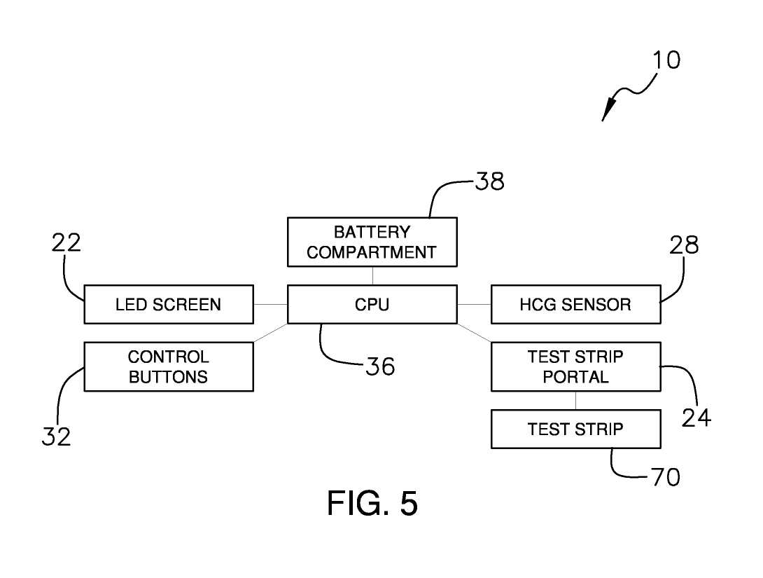

FIG. 5 is a bock diagram view.

### DETAILED DESCRIPTION OF THE DRAWINGS

With reference now to the drawings, and in particular FIGS. 1 through 5 thereof, example of the instant electronic pregnancy test device employing the principles and concepts of the present electronic pregnancy test device and generally designated by the reference number 10 will be described.

Referring to FIGS. 1 through 5 a preferred embodiment of the present electronic pregnancy test device 10 is illustrated.

The present electronic pregnancy test device has been devised to enable a user to regularly monitor levels of human Chorionic Gonadotropin hormone (“hCG”) to test for pregnancy and to ensure appropriate levels of hCG signaling a healthy pregnancy, without the need of visiting a medical practitioner in person to effect the same.

A user deposits a blood sample—typically a drop of blood drawn from a finger tip—upon a test strip which is then loaded into a test strip portal disposed upon the electronic pregnancy test device. Once inserted into the test strip portal, the blood sample is read by an hCG sensor. The level of hCG in the blood sample detected by the hCG sensor is displayed upon a Light Emitting Diode (“LED”) display, said level writable as data to a read-write memory, whereby doubling rates of hCG are determinable and calculable by the present electronic pregnancy test device. A state of pregnancy is thus ascertainable, and the progress of said pregnancy chartable, whereby irregularities in the pregnancy (such as might signify an ectopic pregnancy or a miscarriage, for example) can be elucidated and medical attention sought.

The present electronic pregnancy test 10, therefore, includes an LED display 22 disposed upon a housing 20. A test strip portal 24 is disposed upon an edge of the housing 20, said test strip portal 24 configured to slidingly receive a test strip 70 therein when said test strip 70 is placed upon a test strip support 26 and moved from an extended position to an internal position, whereby the test strip 70 is borne into the housing 20 and engaged by an hCG sensor 28 disposed within the housing 20.

A raised member 30 is disposed upon the test strip support 26, said raised member 30 abutting endwise said test strip 70 when positioned upon the test strip support 26. The raised member 30 ensures the test strip 70 is maintained in an appropriate position upon the test strip support 26 and activates the hCG sensor 28 when the test strip support 26 is moved to the internal position.

For control of the device 10, to input and select between data, a plurality of control buttons 32 is included. The plurality of control buttons comprises a first scroll button 32a configured to moveably select between data displayed on the display 22, a second scroll button 32b configured to moveably select between data displayed on the display 22 in a direction opposite the direction enabled by the first scroll button 32a, a plus button 32c enabling a command or data entry, and a minus button 32d disabling a command or data entry. Additional control buttons are contemplated as part of the device 10, said buttons enabling functionality of the device 10 and interface with data stored within the read-write memory 34.

A Central Processing Unit (“CPU”) 36 is disposed within the housing 20 in operational communication with the hCG sensor 28, the LED Display 22, and the plurality of control buttons 32. The read-write memory 34 is accessed by the CPU 36 and a blood sample collected upon the test strip 70 is loadable into the test strip portal 26, into the electronic pregnancy test device 10, to be read by the hCG sensor 28, whereby a hCG test concentration is detectable, a sample history is storable, and hCG doubling times are calculable.

A battery compartment 38 is disposed within the housing 20, wherein at least one battery is connectable in circuit with the CPU 36, hCG sensor 28, the LED display 22, and the plurality of control buttons 32.

What is claimed is:

1. An electronic pregnancy test device comprising:
a Light Emitting Diode display;
a human Chorionic Gonadotropin hormone sensor;
a test strip portal;
a plurality of control buttons;
a Central Processing Unit in operational communication with the human Chorionic Gonadotropin sensor, the Light Emitting Diode Display, and the plurality of control buttons; and
a read-write memory accessed by the Central Processing Unit;
wherein a blood sample collected upon a test strip is loadable into the test strip portal, thence moveable into the electronic pregnancy test device where read by the human Chorionic Gonadotropin sensor, whereby a human Chorionic Gonadotropin test concentration is detectable, a sample history is storable, and human Chorionic Gonadotropin doubling times are calculable.

2. The electronic pregnancy test device of claim 1 further comprising a test strip support disposed upon the test strip portal, said test strip support moveable between an extended position projecting from the test strip portal and an internal position disposed inside the electronic pregnancy test device at the test strip portal.

3. The electronic pregnancy test device of claim 2 wherein the test strip support further comprises a raised member disposed to abut a test strip when positioned upon the test strip support, said raised member engaging against the test strip when the test strip support is moved from the extended position to the internal position whereby the test strip is borne inside the electronic pregnancy test device and a sample of blood collected upon said test strip is engaged by the human Chorionic Gonadotropin sensor.

4. The electronic pregnancy test device of claim 3 wherein the plurality of control buttons comprises:
a first scroll button configured to moveably select between data displayed on the display;
a second scroll button configured to moveably select between data displayed on the display in a direction opposite the direction enabled by the first scroll button;
a plus button enabling a command or data entry; and
a minus button disabling a command or data entry.

5. The electronic pregnancy test device of claim 4 further comprising a battery compartment disposed accessible upon the electronic pregnancy test device wherein at least one battery is storable in circuit with the Central Processing Unit, the display, the plurality of control buttons, and the human Chorionic Gonadotropin sensor.

6. An electronic pregnancy test device comprising:
a Light Emitting Diode display;
a human Chorionic Gonadotropin hormone sensor;
a test strip portal;
a test strip support disposed upon the test strip portal, said test strip support moveable between an extended position, disposed extending from the test strip portal, and an internal position, disposed inside the electronic pregnancy test device;
a raised member disposed upon the test strip support, said raised member abutting endwise a test strip when positioned on the test strip support;
a plurality of control buttons comprising;
a first scroll button configured to moveably select between data displayed on the display;
a second scroll button configured to moveably select between data displayed on the display in a direction opposite the direction enabled by the first scroll button;
a plus button enabling a command or data entry;
a minus button disabling a command or data entry;
a Central Processing Unit in operational communication with the human Chorionic Gonadotropin sensor, the Light Emitting Diode Display, and the plurality of control buttons; and
a read-write memory accessed by the Central Processing Unit;
wherein a blood sample collected upon a test strip is loadable into the test strip portal, thence moveable into the electronic pregnancy test device where read by the human Chorionic Gonadotropin sensor, whereby a human Chorionic Gonadotropin test concentration is detectable, a sample history is storable, and human Chorionic Gonadotropin doubling times are calculable.

7. The electronic pregnancy test device of claim 5 further comprising a battery compartment disposed accessible upon the electronic pregnancy test device wherein at least one battery is storable in circuit with the Central Processing Unit, the display, the plurality of control buttons, and the human Chorionic Gonadotropin sensor.
